(19) United States
(12) Patent Application Publication
(10) Pub. No.: US 2021/0338073 A1
(43) Pub. Date: Nov. 4, 2021

(54) METHOD FOR CONTROLLING THE ILLUMINATION OF OPHTHALMOLOGICAL DEVICES

(71) Applicant: Carl Zeiss Meditec AG, Jena (DE)

(72) Inventors: David GOLZ, Jena (DE); Dietrich MARTIN, Jena (DE)

(73) Assignee: Carl Zeiss Meditec AG, Jena (DE)

(21) Appl. No.: 17/273,727

(22) PCT Filed: Aug. 15, 2019
(86) PCT No.: PCT/EP2019/071935

§ 371 Date: Mar. 4, 2021

(30) Foreign Application Priority Data
Sep. 10, 2018 (DE) 10 2018 215 307.8

(51) Int. Cl. A61B 3/00; A61B 3/12; H05B 47/16

(52) U.S. Cl. CPC A61B 3/0008; H05B 47/16; A61B 3/12

(57) ABSTRACT

A method for controlling the illumination of ophthalmological devices which operate in an observation mode and a recording mode. The method adapts the illuminance from ophthalmological devices which include an observation mode and a recording mode and the illuminance therefrom being increased above a specified value for the radiant flux Φₛₒₗₗ for the duration of the recording mode. According to the invention, the illuminance is increased to a radiant flux above the specified value Φₛₒₗₗ for the recording mode and lowered to a radiant flux below the specified value for the radiant flux Φₛₒₗₗ for a specified period of time after the recording mode has finished. Although the proposed method for adapting the illuminance is provided for slit lamp microscopes, it can be used for any ophthalmological device that has both an observation mode and a recording mode to obtain high-quality, well-lit recordings in the recording mode.

### RELATED APPLICATIONS

This application is a National Phase entry of PCT Application No. PCT/EP2019/071935 filed Aug. 15, 2019, which application claims the benefit of priority to DE Application No. 10 2018 215 307.8 filed, Sep. 10, 2018, the entire disclosures of which are incorporated herein by reference.

### TECHNICAL FIELD

The present invention relates to a method for controlling the illumination of ophthalmological devices which comprise an observation mode and a recording mode.

### BACKGROUND

According to the known prior art, the radiant flux of the illumination source is increased during the recording mode in such ophthalmological devices, such as, e.g., slit lamps, fundus cameras or the like, in order to generate high-quality, well-lit recordings.

WO 2012/169416 A1 describes a corresponding slit lamp microscope. To this end, the slit lamp microscope comprises an optical illumination system, an optical examination and imaging system, and a control unit. The illumination system is controlled by the control unit in such a way that continuous illumination is provided for the examination and pulsed illumination is provided for the imaging. Here, the pulsed illumination is preferably synchronized with the imaging system in order to be able to capture images of the eye during the examination. Since an LED is used as an illumination system in the proposed case, only the current flow needs to be controlled. Moreover, the control unit is able to capture and/or set the maximum luminous energy in order to observe safety standards.

A further system and method for controlling the light source of a slit lamp microscope is described in CN 102755149. For the eye examination, the light source is set to a brightness level that is comfortable for the eyes in this case. By contrast, the brightness of the light source is set to a higher value during the image recording. During the image recording, the maximum brightness of the light source corresponds to the exposure time of the camera. Once the image recording has finished, the brightness is reduced back to the examination brightness by the control unit.

In the methods for controlling the illumination of a slit lamp microscope known from the prior art, the radiant flux is set to a specified value Φₛₒₗₗ for the purposes of examining the eye.

The radiant flux is increased to a maximum radiant flux Φₘₐₓ permissible by standards for image recording purposes and subsequently reduced back to the specified value for examining the eye Φₛₒₗₗ.

To this end, FIG. 1 shows the time profile of the radiant flux Φ when examining and recording the image of an eye, where
Φₛₒₗₗ denotes the specified value of the radiant power for examination purposes,
Φₘₐₓ denotes the maximum radiant power permissible by standards for the purposes of recording an image of an eye,
Φᵣₑₛ denotes the resultant, mean radiant flux of the treatment,
t₀ denotes the time at the start of the treatment,
t₁ denotes the time at which the image recording starts,
t₂ denotes the time at which the image recording ends, and
t₄ denotes the time at which the treatment ends.

Increasing the radiant flux during the image recording is advantageous in that the quality of the image recordings is significantly improved.

However, this is disadvantageous to the effect that the mean radiant flux Φᵣₑₛ considered over the entire treatment duration from t₀ to t₃ is greater than the specified value Φₛₒₗₗ.

The extent to which the resultant, mean radiant flux Φᵣₑₛ deviates from the specified value Φₛₒₗₗ depends, firstly, on the ratio Φᵣₑₛ:Φₛₒₗₗ and, secondly, on the ratio of the duration of the [symbol] recording (t₂-t₁) to the duration of the treatment (t₀ t₄).

Embodiments of the present invention remedy the disadvantages of the solutions known from the prior art and in which the mean, resultant radiant flux considered over the entire treatment does not exceed the specified value for the radiant flux, which was set as comfortable for the eyes.

Example embodiments of the method for adapting the illuminance from ophthalmological devices comprising an observation mode and a recording mode, in which the illuminance is increased above a specified value for the radiant flux Φₛₒₗₗ for the duration of the recording mode achieves this by virtue of the illuminance for the recording mode being increased to the maximum radiant flux Φₘₐₓ permissible by standards and lowered to below the specified value for the radiant flux Φₛₒₗₗ for a specified period of time after the recording mode has finished.

In accordance with advantageous example configurations, the duration of the increase of the illuminance to the maximum radiant flux Φₘₐₓ permissible by standards corresponds to the exposure time of the employed recording unit and is for example synchronized with the exposure time of the employed recording unit.

According to the invention, the absolute value and the duration of the reduction to below the specified value for the radiant flux Φₛₒₗₗ is dimensioned in such a way that the mean resultant radiant flux Φᵣₑₛ, when considered over the entire treatment duration, corresponds to the specified value Φₛₒₗₗ.

Although the proposed method for adapting the illuminance is predominantly provided for slit lamp microscopes, it can be used, in principle, for any ophthalmological device that comprises both an observation mode and a recording mode, with high-quality, well-lit recordings being intended to be realized in the recording mode.

### BRIEF DESCRIPTION OF THE DRAWINGS

The invention is described in more detail below on the basis of example embodiments. In this respect:

FIG. 1: depicts the time profile of the radiant flux Φ during the treatment of an eye according to known methods and

FIG. 2: depicts the time profile of the radiant flux Φ during the treatment of an eye for the proposed method.

### DETAILED DESCRIPTION

Example embodiments of the invention adapt the illuminance from ophthalmological devices which comprise an observation mode and a recording mode and the illuminance therefrom being increased above a specified value for the radiant power Φₛₒₗₗ for the duration of the recording mode.

According to Example embodiments of the invention, the illuminance is increased to a radiant flux above the specified value Φₛₒₗₗ for the recording mode and lowered to a radiant flux Φₘᵢₙ below the specified value for the radiant flux Φₛₒₗₗ for a specified period of time after the recording mode has finished.

According to the first example configuration, the duration of the increase of the illuminance to a radiant flux above the specified value Φₛₒₗₗ corresponds to the exposure time of the employed recording unit.

Here, it is particularly advantageous, for example, if the duration of the increase in the illuminance is synchronized with the exposure time of the employed recording unit.

In accordance with a second example configuration, the radiant flux above the specified value Φₛₒₗₗ corresponds to the maximum radiant flux Φₘₐₓ permissible by standards.

In accordance with an example configuration, the absolute value and the duration of the reduction to a radiant flux Φₘᵢₙ below the specified value for the radiant flux Φₛₒₗₗ is dimensioned in such a way that the mean radiant flux Φᵣₑₛ, when considered over the entire treatment duration, corresponds to the specified value Φₛₒₗₗ.

Here, the radiant flux is lowered to a minimum Φₘᵢₙ or else to 0 in order to minimize the duration until the ratio Φᵣₑₛ:Φₛₒₗₗ=1 is reached.

To this end, FIG. 2 shows the time profile of the radiant flux Φ when examining and recording the image of an eye using the method according to the invention, where
Φₛₒₗₗ denotes the specified value of the radiant power for examination purposes,
Φₘₐₓ denotes the maximum radiant power permissible by standards for the purposes of recording an image of an eye,
Φₘᵢₙ denotes the minimum, lowered radiant flux,
Φᵣₑₛ denotes the resultant, mean radiant flux of the treatment,
t₀ denotes the time at the start of the treatment,
t₁ denotes the time at which the image recording starts,
t₂ denotes the time at which the image recording ends,
t₃ denotes the time at which the treatment can be continued and
t₄ denotes the time at which the treatment ends.

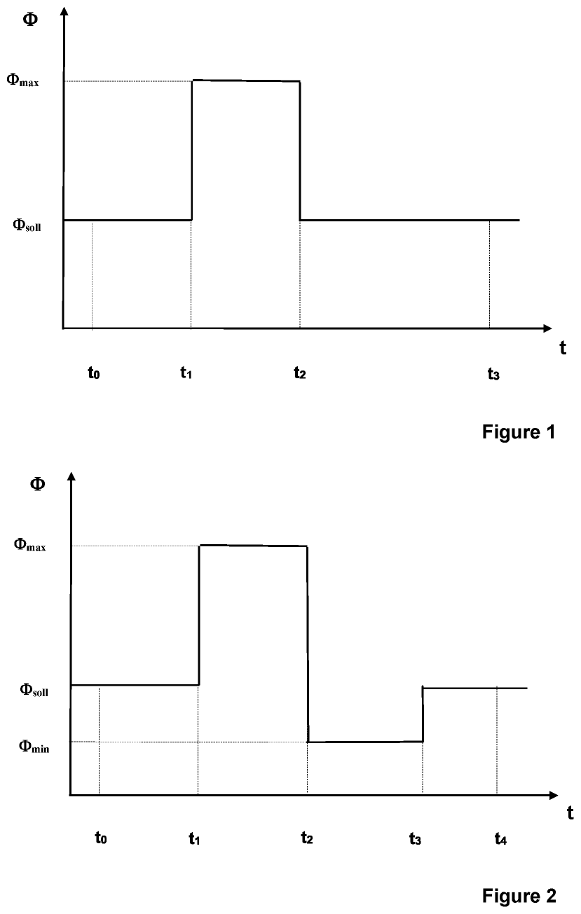

In contrast to the time profile of the radiant flux Φ shown in FIG. 1, the radiant flux Φ is lowered to a radiant flux Φₘᵢₙ below the specified value for the radiant flux Φₛₒₗₗ at the time t₂ (at which the image recording ends) and increased again to the specified value for the radiant flux Φₛₒₗₗ at the time t₃ such that the treatment can be continued.

When satisfying the condition that the mean radiant flux Φᵣₑₛ corresponds to the specified value Φₛₒₗₗ when considered over the entire treatment duration, the ratio Φᵣₑₛ:Φₛₒₗₗ and the ratio of the duration of the image recording (t₂-t₁) to the [symbol] of the treatment (t₀ t₄) should also be taken into account here.

Here, the ratio between t₂-t₁ and t₃-t₂ is decisive under the assumption that the radiant flux corresponds to the specified value in the time periods t₀ to t₁ and t₃ to t₄. Accordingly, the excess radiant flux t₂-t₁ in the time period must be compensated with less radiant flux during the time period t₃-t₂.

Moreover, it is necessary to take account of the duration of the lowering of the image recording (t₃-t₂) to the radiant flux Φₘᵢₙ and the ratio thereof to the specified value for the radiant flux Φₛₒₗₗ.

In detail, the illuminance is only increased again once the resultant, mean radiant flux of the treatment Φᵣₑₛ is equal to the specified value for the radiant flux Φₛₒₗₗ, and the imaging system has completed the processing of the recorded images and is ready for use again.

By reactivating the light sources, the device indicates to the user a renewed readiness for examination or image recording.

In accordance with a further example configuration, the changes in the radiant flux Φ are implemented in the form of rectangular pulses. This can also be gathered from FIGS. 1 and 2.

For example, the changes in the radiant flux Φ can also be implemented in the form of rectangular pulses consisting of a plurality of levels of different power. By way of example, this is advantageous if a series of images should be recorded.

However, the changes in the radiant power Φ can also be implemented in the form of ramp functions.

The solution according to the invention provides a method with which the illuminance from ophthalmological devices can be adapted to the observation mode or recording mode.

In particular, it is possible to increase the illuminance above a specified value for the radiant flux Φₛₒₗₗ for the duration of the recording mode, with the illuminance being increased to the maximum radiant flux Φₘₐₓ permissible by standards for the recording mode and, following the end of the recording mode, being lowered to a radiant flux Φₘᵢₙ below the specified value for the radiant flux Φₛₒₗₗ for a specified period of time.

The present invention ensures that the resultant, mean radiant flux Φᵣₑₛ does not exceed a specified value for the radiant flux Φₛₒₗₗ, set so as to be comfortable for the eyes, when considered over the entire treatment.

The disclosed method is applicable to all ophthalmological devices that comprise an observation mode and also a recording mode and that are intended to be used to realize high-quality, well-lit recordings in the recording mode.

1.-9. (canceled)

10. A method for adapting the illuminance from ophthalmological devices having an observation mode and a recording mode, the method comprising:
increasing the illuminance above a specified value for a radiant flux Φₛₒₗₗ for a duration of the recording mode; and
lowering the illuminance to a radiant flux Φₘᵢₙ below the specified value for the radiant flux Φₛₒₗₗ for a specified period of time after the recording mode has finished.

11. The method as claimed in claim 10, further comprising maintaining the duration of the increase of the illuminance to the radiant flux above the specified value Φₛₒₗₗ to correspond to the exposure time of an employed recording unit.

12. The method as claimed in claim 10, further comprising maintaining a radiant flux above the specified value Φₛₒₗₗ to correspond to a maximum radiant flux Φₘₐₓ permissible by standards.

13. The method as claimed in claim 12, further comprising synchronizing the duration of the increase in the illuminance to the maximum radiant flux Φₘₐₓ permissible by standards with the exposure time of an employed recording unit.

14. The method as claimed in claim 10, further comprising dimensioning an absolute value and a duration of the reduction in the radiant flux Φₘᵢₙ below the specified value for the radiant flux Φₛₒₗₗ in such a way that the mean radiant flux Φᵣₑₛ, when considered over the entire treatment duration, corresponds to the specified value Φₛₒₗₗ.

15. The method as claimed in claim 10, further comprising lowering the radiant flux Φₘᵢₙ to a minimum or to 0 to minimize a duration until the increase to the radiant flux Φₛₒₗₗ.

16. The method as claimed in claim 10, further comprising implementing the changes in the radiant flux Φ in the form of rectangular pulses.

17. The method as claimed in claim 10, further comprising implementing the changes in the radiant flux Φ in the form of rectangular pulses comprising a plurality of levels of different power.

18. The method as claimed in claim 16, further comprising implementing the changes in the radiant flux Φ in the form of ramp functions.
